(19) United States
(12) Patent Application Publication
(10) Pub. No.: US 2004/0193101 A1
(43) Pub. Date: Sep. 30, 2004

(54) Drain assembly for removing liquid from a gas directing tube

(75) Inventors: David Theron Van Hooser, Salt Lake City, UT (US); Cameron Garrett Rouns, South Jordan, UT (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/397,010

(22) Filed: Mar. 25, 2003

(51) Int. Cl. A61M 1/00

(52) U.S. Cl. 604/35

(57) ABSTRACT

An apparatus and method for the removal of condensate from an operational gas directing conduit. Unlike prior devices, the apparatus and method of the present invention provide for condensate removal along a gas directing conduit or system at points other than between gas directing conduit connections or at terminal points of the gas directing conduit. Periodic aspiration of accumulated liquid from the apparatus without interruption of gas flow through the gas directing conduit may also be facilitated.

### BACKGROUND OF THE INVENTION

There are numerous instances in which it would be desirable to transport various gases, aerosols, or vapors through a tube; however, the condensation which can occur in many of those tubes makes such transportation less efficient. This is especially true in many medical applications, such as breathing circuits and the like.

In mechanical ventilator and anesthesia devices employed in patient breathing circuits for a variety of circumstances and reasons, various gases, aerosols or vapors are delivered to the patient. For instance, mechanical ventilators are used to fully provide or augment respiratory gas flow in circumstances in which the patient may be needing ventilatory assistance. In these apparatuses, the breathing frequency, inspiratory, expiratory, and other phases of ventilation can be controlled and varied by manipulation of the controls on the apparatus to meet the individual needs of the patient. Different ventilators may be employed depending upon the condition of the patient: assisted ventilation mode is used for patients who have spontaneous respiration but who may have inadequate alveolar ventilation; whereas controlled ventilation mode is used for those patients with few or no spontaneous respiratory efforts. These ventilators will inflate the patient's lungs with gas under pressure from the ventilator until either a pre-set pressure or pre-set volume, depending upon choice of mode, is achieved. As this pre-set level is reached, inspiration ends and expiration begins.

A typical mechanical ventilator has an inspiration limb for supplying breathing gases to the patient as well as an expiration limb for receiving breathing gases from the patient. The inspiration and expiration limbs are each connected to arms of a Y-connector. A patient limb extends from a third arm of the Y-connector to an artificial airway or face mask for the patient.

Another common type of apparatus is the anesthesia machine, which recirculates the expired breathing gases of the patient in the expiration limb through a CO₂ absorber back to the inspiration limb for rebreathing by the patient. Such a closed breathing circuit prevents loss of anesthetic agents to the ambient air. Such breathing circuits are often operated in a “low flow” mode in which, at least in principle, the amount of fresh, dry breathing gases added to the breathing circuit is in principle only that necessary to replace the gases consumed by the patient.

On both the anesthesia machine and the mechanical ventilator, the inspiration side may monitor and/or regulate such parameters as oxygen percentage and humidity of the air in addition to the volume and frequency of inspiration. All mechanical ventilator and anesthesia machines have the ability to provide active humidification from a humidifier installed on the machine and congruent with the circuit. This active humidifier serves to warm and moisten the gas being delivered to the patient through the circuit. Most mechanical ventilators include an expiration side which receives and filters the exhaled air. The expiration side may be used to monitor the volume of expired air and to control ventilation of the patient. General examples of ventilating systems are contained in U.S. Pat. Nos. 3,090,382, issued to Fegan, et al. on May 21, 1963; U.S. Pat. No. 3,646,934, issued to Foster on Mar. 7, 1972; and U.S. Pat. No. 4,080,103, issued to Bird on Mar. 21, 1978.

Similar breathing circuits are used on patients with chest diseases who receive Intermittent Positive Pressure Breathing (IPPB) treatments of continuous flow aerosol therapy. An IPPB treatment may include the delivery of aerosolized medications to the patient from a nebulizer within the patient breathing circuit under pressure. In addition delivery of the continuous flow aerosol is sometimes made to the patient by virtue of a open, unpressurized breathing circuit, from a large volume nebulizer.

One of the problems which occur in both pressurized (e.g., mechanical ventilator, anesthesia, and IPPB) circuits and un-pressurized (continuous flow aerosol) delivery systems involves the undesirable collection of condensate generally in the tubing which extends from the control apparatus (e.g., mechanical ventilator, anesthesia machine, IPPB or large volume nebulizer) to the patient. While it is desirable that the subject inspire moist, warm breathing gases, the presence of such humidity within the breathing circuit does have disadvantages. In addition, as the warm, moist exhaled gas from the subject is at body temperature as it passes through the breathing circuit, which is at room temperature, the water vapor in the exhaled gas condenses within the inner walls and upon components of the breathing circuit. As the breathing of the subject continues, the condensed water (“rain out”) accumulates. The accumulated water may interfere with the operation of valves, sensors and other components, or the flow of gas through the breathing circuit. It may also become a medium for microbiological growth within the circuit and is considered to be bio-hazardous waste. Such accumulations therefore present a problem especially in closed circuit breathing systems.

The rain out concerns arise for a number of reasons including the fact that gases saturated with water vapor are passed through highly sensitive components in the circuit. The condensed moisture collects on these components and can effect the ventilator function, and may cause damage to certain ventilator components.

The condensation of water vapor can present additional problems to the operation of the ventilator. For example, the ventilator can include a flow transducer which is used to determine the volume of gas expired by the patient. This transducer may take the form of a fine mesh screen which provides resistance to air flow. The increase in pressure resulting as the exhaled gas encounters the screen is used to determine the volumetric flow. If moisture accumulates on the screen, this will present an additional barrier to air flow, thereby resulting in false readings of the flow transducer. The air pressure downstream of the flow transducer also is used in certain instances to trigger the delivery of inspiration air to the patient. In operation, the development of negative pressure downstream of the flow transducer signals the beginning of inspiration by the patient, and the ventilator acts in response thereto. Condensate on the flow transducer screen may adversely affect this function. Also, a bacteria filter may be provided in the expiration line from the patient to prevent the transmission of bacteria into the room. The collection of moisture on the bacteria filter will present a greater resistance to flow, affecting both the accuracy of the volumetric flow readings as well as the ease with which the patient may exhale. It is apparent that the condensation of water vapor at the filter or within the ventilator may significantly affect the desired operation of the ventilator in all of these respects.

Another problem is that the rain out (unwanted accumulation of condensed water vapor) may also physically interfere with flow of gas from the anesthesia machine, ventilator or large volume nebulizer. In the case of the ventilator it may effect the pressures delivered, or the alarm thresholds when it interferes (reduces) the flow. On the anesthesia machine it may also effect flow and gas composition because the anesthetic vapors may be “washed out” of the inspired gas because of the condensation. Most large volume nebulizers utilize a venturi to mix room air with the oxygen powering the nebulizer. Because the circuit is open, accumulation of water in the circuit can cause reduced flow, which in turn reduces the efficiency of the nebulizer causing the oxygen percentage to be higher than desired.

Various solutions have been proposed to remedy this problem, but so far have not been entirely successful. For instance, water traps or drains, such as those disclosed in U.S. Pat. Nos. 5,722,393 to Bartel et al., 4,867,153 to Lorenzen et al., and 4,327,718 to Cronenberg may be inserted in the breathing circuit in an effort to prevent water from reaching critical components.

Unfortunately, most of the known water traps must be drained frequently, often times necessitating the breaching of the closed circuit while the water is discarded. Further some of these water traps are very large and increase the compressible volume of the patient circuit. Some of the smaller volume water traps tend to lose effectiveness by dumping water back into the patient circuit if the patient should move or pull the tubing. Of course, the smaller volume traps also must be drained more frequently. When draining occurs in most of the existing water traps, the patient circuit, if operating under pressure, must be shut down and depressurized while the water is being emptied from the trap. Of course, during the depressurized condition, delivery of the gas, air or vapors to the patient is interrupted. The necessity of manual draining of the existing traps is time consuming and requires periodic monitoring by the attendants to observe when the trap is becoming filled. In addition, the interruption of the service to the patient during depressurization of a pressurized patient breathing circuit is undesirable.

While two water traps or drains identified above (U.S. Pat. Nos. 4,327,718 and 4,867,153) are known to allow draining without the need to depressurize the patient circuit, both undesirably require the insertion of the trap (having or necessitating a Y-connector) between two pieces of the circuit and are not contemplated for use with a circuit having heated wires throughout because of the need for the Y-connector to be inserted between pieces of the circuit. Further, U.S. Pat. No. 4,327,718 undesirably requires a gas-impervious material which prevents anything but water from entering the drain; however, the device still requires an attendant to periodically exchange a collection container, thereby potentially subjecting the attendant to potential exposure to biohazardous fluids or waste.

Other proposed solutions to the condensation problem include providing for one or more portions of the breathing circuit particularly affected by moisture accumulation to be heated in an attempt to prevent condensation of the water vapor. This may be carried out, for example by resistance heaters, such as wires that are wrapped around the tubing of the limbs, and around valves, etc. An example of such a respiratory humidifier conduit incorporating a heating wire is disclosed in U.S. Pat. No. 5,537,996 issued to McPhee on Jul. 23, 1996. The heating wire disclosed is a looped heating element with the two free ends of the loop emerging from one end of the conduit for connection to a source of alternating voltage on the humidifier. This form of heated conduit where the heating wire lies in a random path along the bottom of the conduit has the disadvantage that gases passing through the conduit are not uniformly heated across the width of the conduit. In addition, the random nature of the wire's distribution allows for localized regions of the conduit walls to be at a temperature sufficiently low so as to allow condensation or rain out to occur while other areas are heated excessively.

Other humidifier conduits have a heating wire wound around the outside of the conduit in an attempt to evenly apply heat to the conduit wall (both around the conduit and along the length of the wall) to overcome the problem of condensation. Examples of externally wound heated humidifier conduit may be seen in U.S. Pat. No. 4,686,354 issued to Makin on Aug. 11, 1987 and U.S. Pat. No. 5,357,948 issued to Eilentropp on Oct. 25, 1994. Both of these configurations, however, require the power drawn by the heating element to be sufficient to transmit heat through the conduit walls and into the gases. Accordingly, the power drawn by the heater wire is excessive as is the temperature of the wire. In addition, as heat from the heater wire must first pass through the conduit wall, the time taken to heat the gases is excessive, and the temperature of the outer surface of the conduit could be high enough to burn a patient or care giver, thereby creating an hazard.

As noted above, while heating can in some instances delay the onset of condensation and avoid or reduce condensation in critical parts of the circuit, it is difficult or impossible to fully prevent precipitation of water vapor out of the breathing gases. This is especially true as the environment in which the circuit is used is frequently different with each use.

The few solutions to the rain out problem have offered limited success. Thus, a need remains for a component or ventilating system which avoids the problems associated with condensation of vapor in a gas transport tube or conduit.

### SUMMARY OF THE INVENTION

In response to the difficulties and problems discussed above, a fluid collection device for collecting condensed vapor and moisture from a gas directing conduit having an opening created therein has been developed. More specifically, one aspect of this invention is directed to a device including a lid member; a reservoir capable of forming a seal with the lid member, the reservoir intended for receiving vapor and moisture; and a duct having a proximal end and a distal end, the distal end being in communication with the lid member and the proximal end adapted for communication with the conduit

A second aspect of the present invention is directed to a method of draining fluid from a gas directing tube including: providing a fluid collection device; creating an opening in the gas directing tube; and inserting at least a portion of the fluid collection device into the opening in the gas directing tube such that fluids within the gas directing tube may flow into the reservoir.

Another aspect of the present invention is directed to a method of draining a heated wire circuit. Specifically, the method includes providing a fluid collection device intended for use with a circuit having an opening created therein, the collection device including: a lid member; a reservoir capable of forming a seal with the lid member, the reservoir intended for receiving vapor and moisture; a conduit having a proximal end and a distal end, the distal end being in communication with the lid member and the proximal end adapted for communication with the circuit; and a retention mechanism for maintaining the position of the assembly relative to the circuit; creating an opening in the circuit such that at least a portion of the conduit may be inserted therein without interfering with the heating elements of the circuit; and securing the assembly to the circuit such that fluids within the circuit may flow into the reservoir.

### BRIEF DESCRIPTION OF THE DRAWINGS

FIG. 1 is a perspective view illustrating a prior art embodiment of a patient breathing circuit.

FIG. 2 is a schematic showing another known ventilating system.

FIG. 3 is a side view of one embodiment of the present invention in contact with a gas directing tube, the gas directing tube being shown in cross-section.

FIG. 4 is a side view of another embodiment of the present invention, the gas directing tube being shown in cross-section.

FIG. 5 is a schematic of the embodiment shown in FIG. 3 (shown without the retention mechanism 106) and an aspiration mechanism.

FIG. 6 is another view of a portion of the aspirating mechanism of FIG. 5.

FIG. 7 is a cross-sectional view of the probe of FIG. 5 positioned in a probe-receiving assembly.

FIG. 8 is a side view of the embodiment of FIG. 3 shown with a retention mechanism in its closed position.

FIG. 9 is a schematic of a ventilating circuit having two drain assemblies of the present invention therein.

### DETAILED DESCRIPTION OF THE ILLUSTRATED EMBODIMENTS

Reference will now be made to the drawings in which the various elements of the present invention will be given numeral designations and in which the invention will be discussed so as to enable one skilled in the art to make and use the invention. Like numerals are used to designate like parts throughout. It should be appreciated that each example is provided by way of explaining the invention, and not as a limitation of the invention. For example, features illustrated or described with respect to one embodiment may be used with another embodiment to yield still a further embodiment. These and other modifications and variations are within the scope and spirit of the invention.

The following detailed description will be made in the context of a ventilation system or breathing circuit which is adapted for medical use. It is readily apparent, however, that the article of the present invention would also be suitable for use with other types of systems, circuits or conduits and the like and is not intended to be limited to medical devices or use in a medical field.

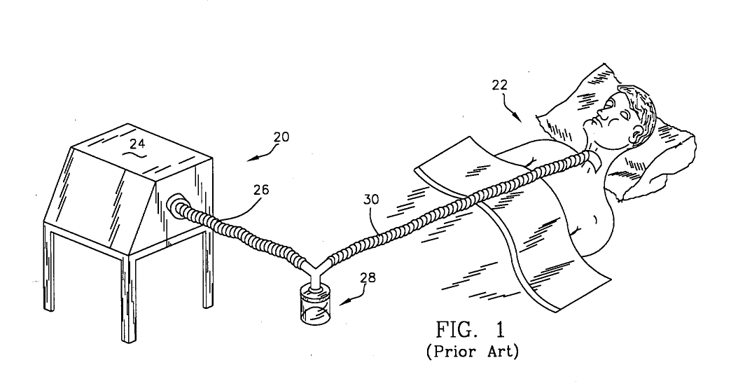

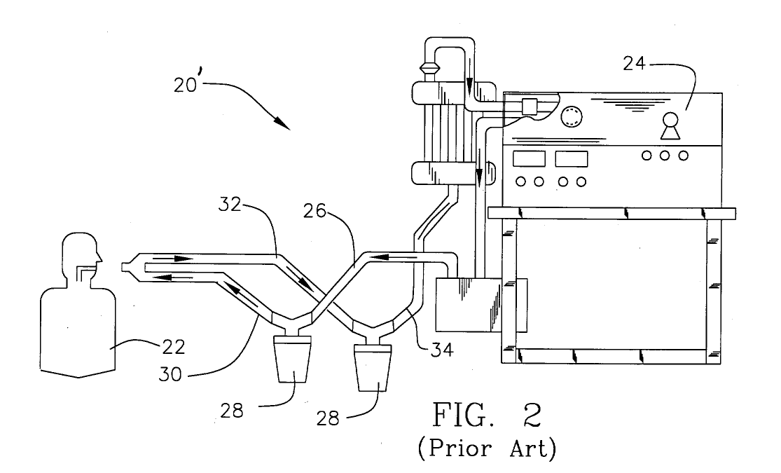

Turning now to the drawings, and FIG. 1 in particular, there is illustrated a patient breathing apparatus 20 as it may appear during use with a patient 22. Apparatus 20 consists generally of three components: a controllable patient breathing device 24, a length of flexible tubing 26 extending from the breathing device 24 to a water trap 28 and another section of flexible tubing 30 extending to patient 22. The apparatus 20 may only have an inspiration side as in FIG. 1 or it may be similar to the set up shown in FIG. 2 in which the apparatus 20′ also includes an expiration side which receives exhaled breathing gases from the patient 22 and returns the gases to breathing device 24 for recirculation. A second water trap 28 may be included along the expiration side of the apparatus 20′ between sections of flexible tubing 32 and 34. While apparatus 20 and 20′ may contain other components, such as various flow sensors and pressure sensors, check valves, heaters, air coolers, and the like, which can effect the amount of moisture or condensation in the system, these components are not necessary to understand the disclosure herein and thus need not be discussed as their inclusion within the scope of the invention will be appreciated by those having skill in the art.

Patient breathing device 24 may be any of the well-known devices for delivering gases, vapors, air or the like to a patient in applications such as ventilation, inhalation, anesthesia and respiratory therapy. For instance, patient breathing device 24 may be a mechanical ventilator, either of the pressure-pre-set or the volume-pre-set types. In controllably delivering fluid, e.g., under pressure to the patient, this breathing device either includes a source of fluid (not shown) within or is connected to such a source so that it may be passed on to the patient. In addition, a typical ventilator allows the breathing frequency and inspiratory and expiratory phases of ventilation to be varied to meet the individual needs of the patient, with a variety of settings available to the attendant in establishing the correct breathing rhythm each time the ventilator is used.

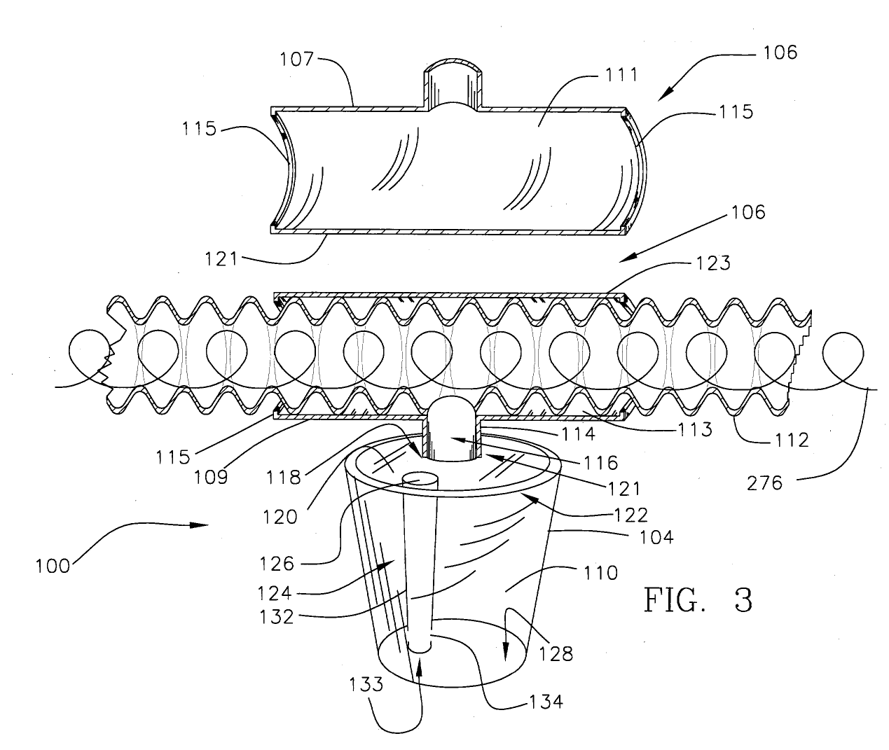

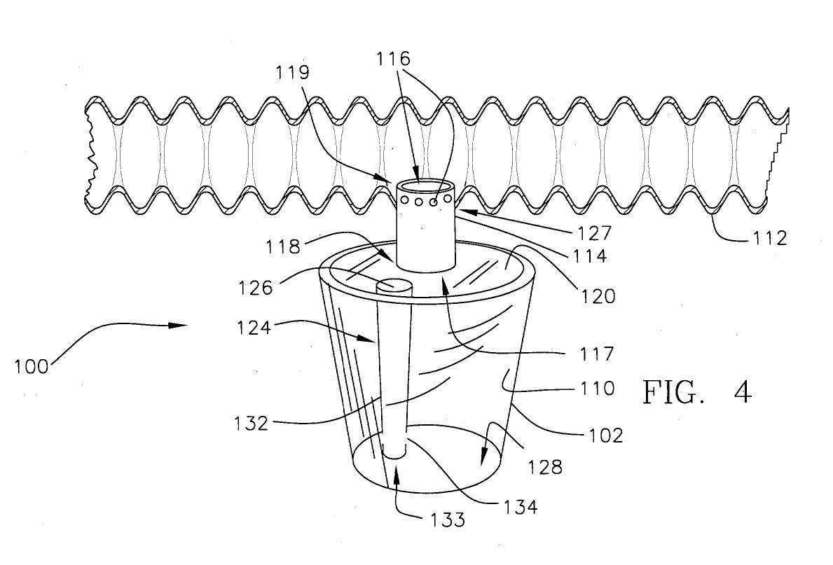

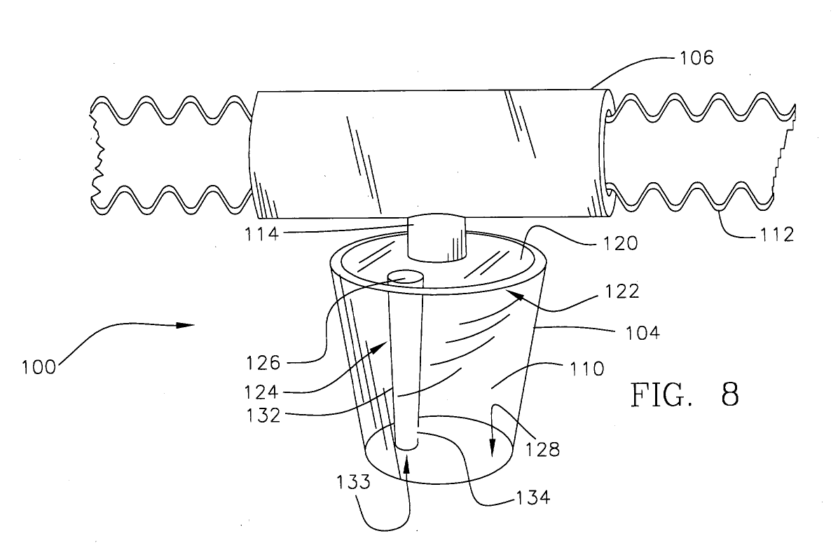

Referring now to FIGS. 3 and 4, there is shown a fluid collection device or drain assembly 100 made in accordance with the teachings of the present invention. The drain assembly is intended for collecting condensed vapor and moisture from a gas directing conduit 112 having an opening 127 (FIG. 4) created therein. The drain assembly 100 includes a lid 120, a reservoir 104 capable of forming a seal with the lid 120, a duct 114 having a proximal end 119 (FIG. 4) and a distal end 121 (FIG. 3), the distal end 121 being in communication with the lid 120 and the proximal end 119 adapted for communication with the conduit 112. Collection reservoir 104 is shown as having an interior 110 (FIG. 3) and being in fluid communication with the conduit 114. The drain assembly 100 may further include a retention mechanism 106 (FIGS. 3 and 8) capable of retaining or assisting with the retention of the assembly 100 (FIGS. 3-5, 8 and 9), and more specifically duct 114, in position relative to the conduit 112.

It will be appreciated that while reference is made to a gas directing tube or conduit 112 (FIGS. 3, 4 and 9), any suitable channel, circuit, cylinder, duct, hose, pipe, or the like also may be used. However, for ease of reading and understanding of this disclosure, and not intending to be limited thereby, gas directing conduit 112 will hereinafter be referred to as a conduit 112. Similarly while reference is made to a duct 114 (FIGS. 3, 4 and 8), any suitable channel, circuit, conduit, cylinder, hose, pipe, or the like also may be used. However, for ease of reading and understanding of this disclosure, and not intending to be limited thereby, duct 114 will hereinafter be referred to as a conduit 114.

Referring again to FIG. 3, the assembly of the present invention is shown having a conduit 114 which is capable of conducting fluid to the reservoir 104. Such a conduit 114 may be part of the retention member 106, or the conduit 114 may be a separate component or integrally formed with the lid 120. In either instance, conduit 114 is desirably rigid but could be made of a flexible or semi-rigid material.

As shown in FIGS. 3 and 4, conduit 114 has a plurality of openings 116 therein so as to allow fluid to flow through at least a portion of the conduit 114. That is, the conduit 114 is adapted such that once the drain assembly 100 is properly positioned in the conduit 112 at least a portion of conduit 114 is positioned in or about conduit 112 such that conduit 114 provides a way of allowing fluid, especially liquids, in the conduit 112 to flow into conduit 114 and out of the conduit 112. In order to allow the fluid (e.g., liquid and/or gas) to enter the conduit 114 and eventually flow into reservoir 104, one or more openings 116 is desirably positioned on or about the conduit 114 in such a way that when the drain assembly 100 is properly positioned, at least one of the openings 116 is in fluid communication with conduit 112 so as to allow for fluid in conduit 112 to enter the collection reservoir 104. As will be apparent, there must also be at least one opening 117 (FIG. 4) which provides the fluid which enters the conduit 114 the opportunity to exit or flow out of the conduit 114 and into reservoir 104.

While FIG. 4 illustrates an embodiment showing an opening 116 at the proximal end 119 of the conduit 114, it will be appreciated that the orientation of the openings 116 may be such so as to allow for a variety of draining scenarios. For example, the one or more openings 116 in the conduit 114 which are in sufficient proximity to the proximal end 119 of the conduit 114 so as to be within conduit 112 (FIG. 4) when the drain assembly 100 is properly positioned need not be oriented such that all liquid accumulated within the conduit 112 adjacent the drain assembly 100 and specifically the conduit 114 is removed. Instead, the openings 116 may be oriented such that liquid is drained only after a certain level of liquid in the conduit 112 about the conduit 114 is achieved. Further, as suggested above, there may be one or numerous openings (e.g., holes, slots, etc.) 116 in the conduit 114 which allow fluid to enter the conduit 114. The number and/or the size of the openings 116 present can vary and may be varied in design depending on the intended use of the drain. For example, it may be desirable to have a number of smaller openings in one embodiment while it may be desirable to have one large opening in another or, further still, it may be desirable to have a combination of larger and smaller openings as well as openings of different shapes. It will be appreciated that the location of the openings 116 should be such that fluid will not flow into one opening 116 and out another so as to cause or result in the leakage of fluid from the conduit 112 and the collection reservoir 104.

The conduit 114 (FIGS. 3, 4 and 8) will generally be directed in a downward direction and should be in sealed fluid communication with the hollow interior 110 (FIGS. 3, 4 and 8) of the reservoir 104 (FIGS. 3-5 and 8). More specifically, the downwardly directed conduit 114 (FIGS. 3, 4 and 8) is desirably in alignment with an opening 118 (FIGS. 3-5) in the lid 120 (FIGS. 3-5 and 8) of the associated reservoir 104. The hollow conduit 114 (FIGS. 3, 4 and 8) is thus secured to the lid 120 (FIGS. 3-5 and 8) in a secure, sealed relation as by bonding, or in any other suitable manner.

Depending on the embodiment, the associated lid 120 (FIGS. 3-5 and 8) may be force-fit at lip 122 (FIGS. 3 and 8), in a conventional fashion, upon the upper edge of the associated reservoir 104 (FIGS. 3-5 and 8) to releasably secure the two parts together in air-tight relation, or the lid 120 (FIGS. 3-5 and 8) may be more permanently secured to the reservoir 104 in a sealed engagement.

In those embodiments in which it desired to maintain a closed system and/or where it is desired to be able to remove fluids from the reservoir 104 without subjecting an attendant to exposure to the fluids, the drain assembly 100 (FIGS. 3-5, 8 and 9) may have an access port to allow for the aspiration of fluid from the reservoir, generally, or, more specifically, a probe-receiving assembly, generally designated 124 (FIGS. 3-5, 7 and 8). Although the probe-receiving assembly 124 may be in a variety of locations in the reservoir 104, including but not limited to the side or bottom of the reservoir 104, it is shown in FIGS. 3-5, 7 and 8 as being positioned in the lid 120 of the reservoir 104. The embodiments of FIGS. 3, 4 and 8 show a lid 120 having an aperture 126 therein into which the probe-receiving assembly 124 is fitted in air-tight relation. Adhesive may be used, if desired, to secure the probe-receiving assembly 124 in its inserted relation in lid 120 at aperture 126. In addition to the probe-receiving assembly 124 mentioned above and shown in FIGS. 3-5, 7 and 8, it will be appreciated that any other suitable way of aspirating or otherwise removing fluids from the reservoir, including, for example, a suction hose or the like which may be attached to a connection member or fitting (not shown) and which extends from a side of the reservoir 104 is also contemplated by the present invention.

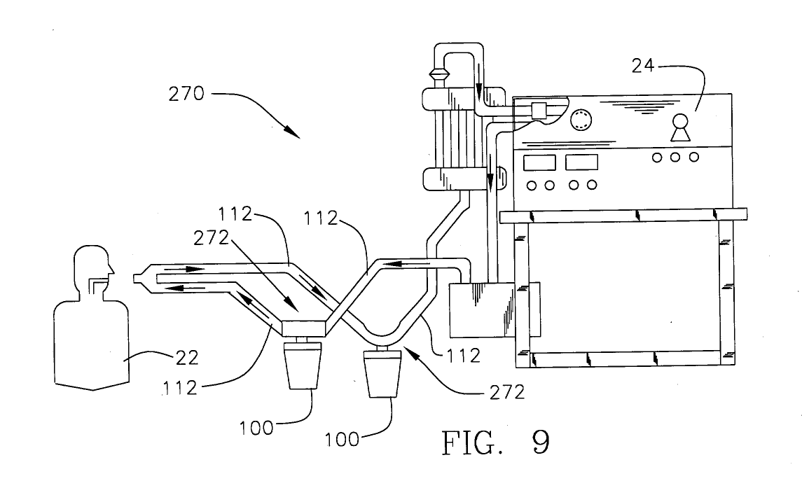

The probe-receiving assembly 124 generally will have a normally closed position to which it defaults so as to prevent contamination of the breathing circuit or ventilating system 270 (FIG. 9) as well as to avoid loss of pressure within the interior of the ventilating system if the system is closed. The normally closed probe-receiving assembly 124 (FIGS. 3-5, 7 and 8) also will help avoid spills or leaks from the drain assembly 100 (FIGS. 3-5, 8 and 9) should the reservoir 104 (FIGS. 3-5 and 8) become filled or be tipped over.

While the drain assembly 100 may be manufactured with components of a variety of materials and sizes, it is desirable that the reservoir 104 be of sufficient size that a substantial quantity of liquid may be accumulated therein, provided it is desirable that the weight of the accumulated liquids therein does not put undo pressure or stress on the ventilating system or its components.

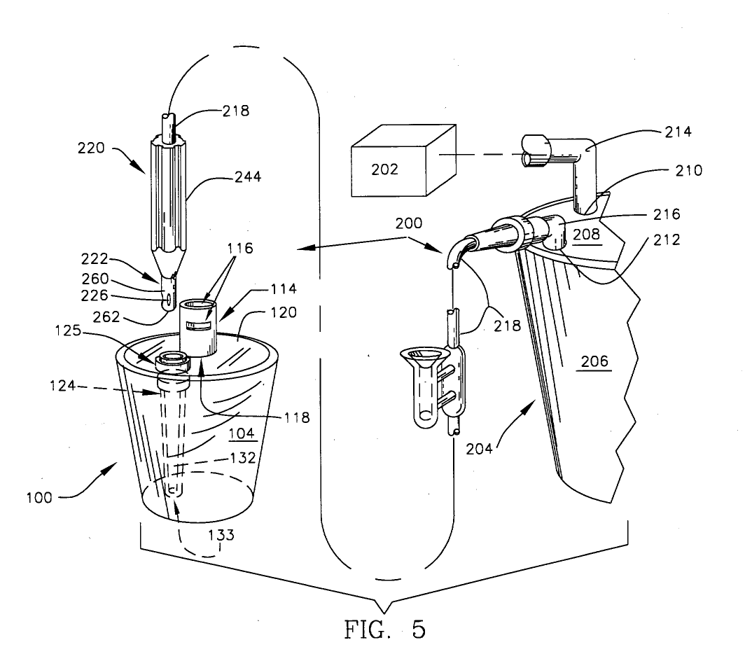

Referring again to the probe-receiving assembly 124 (FIGS. 3-5, 7 and 8), any number of suitable configurations are contemplated for use with the present invention. One such configuration may generally include, in its simplest form, an evacuation pipe 132 (FIGS. 3-5 and 8) which extends from the lower surface of the lid 120 (FIGS. 3-5 and 8) to a location immediately above the bottom surface 128 (FIGS. 3, 4 and 8) of the reservoir 104 (FIGS. 3-5 and 8), whereby, under force of vacuum, essentially all of the liquid contents of the reservoir 104 may be evacuated without ventilation interruption. A number of variations of the probe-receiving assembly 124 are contemplated and include, but are not limited to: the tapering of pipe 132; the inclusion of one or more additional openings 134 (FIGS. 3, 4 and 8) in the pipe 132 near the bottom surface 128 of the reservoir 104 such that removal of the accumulated liquids still may occur if the distal opening 133 (FIGS. 3-5 and 8) of the pipe 132 becomes blocked or clogged; the extension of the pipe 132 above the surface of the lid 120; and/or the inclusion of a cap or adapter 125 (FIG. 5) at or above the surface of the lid 120 which provides for sealed engagement between a variety of different sized and/or shaped probes 220 (FIGS. 5-7) and the probe-receiving assembly 124 (FIGS. 3-5, 7 and 8). It will be appreciated that the additional openings 134 (FIGS. 3 and 4) in the pipe 132 (FIGS. 3-5 and 8) also may provide for or enable turbulent cleaning of the pipe opening 133 (FIGS. 3-5 and 8) which may extend the useful life of the drain assembly 100 (FIGS. 3-5, 8 and 9).

Other variations, as suggested above, include a valve disk (not shown) or the like made of a material having memory characteristics such that when probe 220 (FIGS. 5-7) is removed from the probe-receiving assembly 124 (FIGS. 3-5, 7 and 8), the disk returns to its original or default position so as to allow both the probe-receiving assembly 124 and the ventilating system 270 (FIG. 9) to remain “closed”. Still other variations are contained in U.S. Pat. No. 4,867,153 issued to Lorenzen on Sep. 19, 1989 and assigned to Ballard Medical Products (a subsidiary of the assignee of the present invention), the disclosure of which is incorporated by reference in its entirety. While a number of exemplary variations have been discussed and incorporated by reference numerous other variations exist, will be appreciated, and are contemplated to be within the scope of the present disclosure.

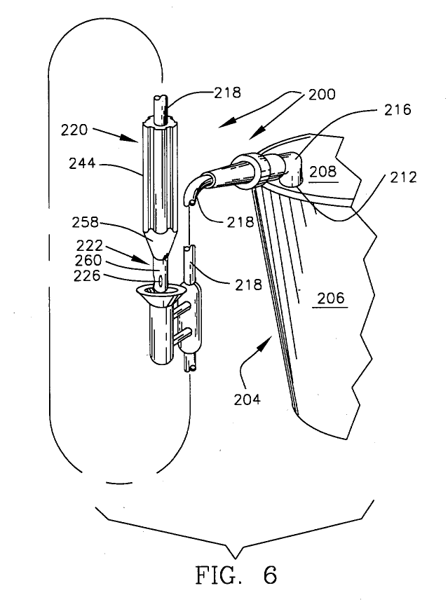

Reference is now made to FIGS. 5 through 7, which illustrate an exemplary closed liquid evacuation system for the removal of liquid accumulated in a drain assembly made and practiced in accordance with the present invention. For example, the evacuation system, which is generally designated 200 in FIGS. 5 and 6, may be used from time to time to remove liquid accumulating in all of the reservoirs 104 of a ventilation system 270 (FIG. 9).

The evacuation system 200, shown in FIGS. 5 and 6, includes a vacuum source 202 (FIG. 5), such as, for example, a hospital suction system, and a large capacity liquid storage bucket 204 comprising a large volume lower receptacle 206 and a press-fit lid 208, both of well-known conventional design. The lid 208 is interrupted by two apertures 210 (FIGS. 5) and 212 (FIG. 5 and 6), each of which is shown in communication with a conduit 214 (FIGS. 5) and 216 (FIG. 5 and 6), respectively. The vacuum generated at source 202 (FIG. 5) is communicated along a conduit 214 (FIG. 5) to the interior of the air-tight bucket 204. The vacuum pressure is communicated from the interior of the bucket 204 through the hollow of the second conduit 216. As shown the conduit 216 is connected to a flexible tube 218 desirably formed of suitable synthetic resinous material. The vacuum vents through the hollow of a distal probe, generally designated 220, at ports 226 located at the distal end or tip 222 of the probe 220.

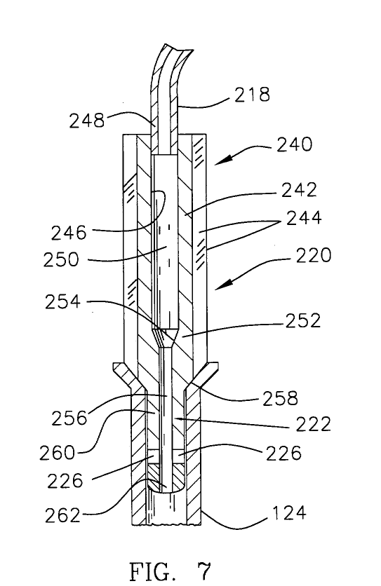

In reference to FIG. 7, the probe 220 itself is illustrated as including an elongated sleeve 240 which includes a wall 242, the exterior surface 244 of which is longitudinally serrated. The wall 242 also comprises an internal annular surface 246. The end 248 of the tube 218 is fitted into and secured at the trailing end of the surface 246, using a suitable bonding agent or adhesive. The wall 242 defines a chamber 250 which is in fluid communication with the interior of the tube 218.

The thickness of the wall 242 is enlarged at site 252 adjacent diagonal internal shoulder 254, which reduces the diameter of the internal vacuum chamber 256 at site 252. The exterior diameter of the probe 220 is also reduced at diagonal internal shoulder 258, which integrally merges with the exterior surface 260 of the probe tip 222. The tip 222 is illustrated as being equipped with two opposed side ports 226. A third axial port 262 also exists at the end of the central passageway through the probe 220.

With the vacuum source 202 (FIG. 5) operating, negative pressure is delivered to the interior of the sealed bucket 204 (FIGS. 5 and 6) and along the hollow interior passageway of tube 218 (FIGS. 5-7) and the hollow chambers 250, 256 (FIG. 7) of the probe 220 (FIGS. 5-7). The probe 220 may be placed in alignment with the probe-receiving assembly 124, as illustrated in FIG. 5. When the tip 222 of the probe 220 is inserted into the interior of the probe-receiving assembly 124, it may assume the position illustrated in FIG. 7. Once the probe 220 (FIGS. 5-7) is properly positioned with probe-receiving assembly 124 (FIGS. 3-5, 7 and 8), the negative pressure contained within the hollow interior of the probe 220 (FIGS. 5-7) is transmitted to the interior of the associated sealed reservoir 104 (FIGS. 3-5 and 8) via the evacuation pipe 132 (FIGS. 3-5 and 8). This then causes liquid accumulated in the reservoir 104 (FIGS. 3-5 and 8) to be evacuated by suction up the evacuation pipe 132 (FIGS. 3-5 and 8), through the probe ports 226 (FIGS. 5-7) and 262 (FIGS. 5 and 6) into the interior chambers 256, 250 (FIG. 7) of the probe 220 (FIGS. 5-7), along the hollow passageway of the tube 218 (FIGS. 5-7), and into the sealed storage bucket 204 (FIGS. 5 and 7).

The probe-receiving assembly 124 (FIGS. 3-5, 7 and 8) may be designed such that the user of the probe 220 (FIGS. 5-7) needs to maintain the probe 220 in the inserted position illustrated in FIG. 7 in order to cause vacuum evacuation of liquid contained in the reservoir 104 (FIGS. 3-5 and 8) to continue. In such an embodiment if, for whatever reason, the user were to remove the force needed to hold the probe 220 (FIGS. 5-7) in the evacuating position, the probe 220 would migrate from the inserted position of FIG. 7 to a non-evacuating position.

Alternatively, the probe-receiving assembly 124 (FIGS. 3-5, 7 and 8) could be designed such that no external pressure or force is needed to maintain the probe 220 (FIGS. 5-7) in an evacuating position. This would allow an attendant to insert the probe 220 (FIGS. 5-7) into the probe-receiving assembly 124 (FIGS. 3-5, 7 and 8) upon initial assembly or hook-up of the ventilating system to the patient and thus not require as regular or as frequent monitoring of the liquid accumulation in the reservoir 104 (FIGS. 3-5 and 8). In such an embodiment, a relatively low amount of suction might be set so as to enable evacuation of accumulated liquid from the reservoir 104 with minimum impact on the efficiency of the system when little or no liquid is present in the reservoir 104. Such an embodiment also would desirably have a normally closed valve, mechanism or the like (not shown) in the probe-receiving assembly 124 (FIGS. 3-5, 7 and 8) such that if or when the probe was dislodged (e.g., intentionally or inadvertently) from the probe-receiving assembly 124 liquid would not leak from the probe-receiving assembly 124.

Referring again to FIGS. 3 and 8, there is shown a retention mechanism 106 for maintaining or assisting in maintaining the position of drain assembly 100 relative to the conduit 112. As illustrated in FIG. 8, the retention mechanism 106 encompasses a portion of the conduit 112 and reduces or prevents the possibility of the drain assembly 100 becoming dislodged from the conduit 112 either from contact or movement or from the weight of the drain assembly, especially as liquid accumulates therein. In FIG. 3 there is shown a two component retention mechanism 106 which interlocks or snaps together at edges 107 and 109, and 121 and 123, respectively. The two components 111 and 113 of the retention mechanism 106 may be hingedly attached or may be completely separably. FIG. 8 illustrates one embodiment where the retention mechanism 106 is in a closed position and FIG. 3 illustrates the same embodiment where the retention mechanism 106 is in an open or partially open position.

It is of further note that in one or more embodiments that the retention mechanism 106 may be, for example, fixed to another component of the drain assembly 100 or the retention mechanism 106 may be removably mounted or secured about conduit 114.

In other embodiments, other types or forms of retention mechanisms are contemplated. For example, clamps (not shown) may be used to secure the drain assembly 100 to the conduit 112. Further still, other mechanisms or ways of encompassing a portion of conduit 112 so as to maintain or assist in maintaining the position of drain assembly 100 relative to the conduit 112 will be appreciated and are contemplated to be within the scope of the present invention.

In addition to the external retention mechanisms described or suggested above, it is also contemplated that an internal retention mechanism such as that identified as 106′ illustrated in FIG. 4 may be used. Such a retention mechanism 106′ could be recessed in or abut against the conduit 114 until positioned within conduit 112 at which time the mechanism 106′ could be activated or triggered so that it would deploy as illustrated.

No matter which type of retention mechanism is utilized, a gasket or other type of seal 115 (FIG. 3) may be used so as to reduce or avoid leakage from conduit 112 (FIGS. 3, 4 and 9). It will be appreciated that such a seal 115 (FIG. 3), for example, could be about a portion of conduit 114 (FIGS. 3, 4 and 8) so as to avoid leakage from the conduit 112. Additionally, depending on the type of retention mechanism utilized, such a seal 115 (FIG. 3) could be placed between the retention mechanism 106 (FIG. 3) and the conduit 112 (FIGS. 3, 4 and 9) such that, even if some leakage between the conduit 112 and conduit 114 (FIGS. 3, 4 and 8) were to occur, the liquid still would not pass outside of the drainage assembly 100.

As noted above any suitable material may also be used for the components of the drain assembly; however, as will be appreciated there may be certain materials which are more desirable in certain embodiments. For instance, the use of certain materials for one or more components of the drain assembly may be more desirable for use with heated wire circuits than those without electrical elements therein as the electrical current flowing through the circuit could cause the heating element to short out or shock the user or attendant if a drain assembly or certain components thereof (e.g., conduit 114) are constructed with conductive materials which in turn come in contact with the heating element. Alternatively, it may be desirable to manufacture a drain assembly with materials which may provide for components of different colors including, for example, those which result in an assembly which is at least in part transparent or substantially so, such as the embodiments shown in FIGS. 3, 4 and 8.

The present invention is also directed to one or more methods of draining accumulated liquid from a gas directing tube or conduit. Specifically, one embodiment is directed to a method for draining a ventilating system 270 as illustrated in FIG. 9. The method includes providing fluid collection device or drain assembly 100 (FIGS. 3-5, 8 and 9) such as the type discussed above; creating an opening 127 (FIG. 4) in the conduit 112 (FIGS. 3, 4 and 9) of the ventilating system 270 (FIG. 9); and inserting at least a portion of the fluid collection device 100 into the opening 127 (FIG. 4) in the conduit 112 such that fluids within the ventilating system 270 and, more specifically the conduit 112, may flow into the reservoir 104 (FIGS. 3-5 and 8) of the drain assembly 100 (FIGS. 3-5, 8 and 9). In one or more embodiments, the drain assembly 100 desirably will be sized such that the conduit 114 (FIGS. 3, 4 and 8) thereof will create a seal between the conduit 112 and the conduit 114. The method of the present invention may also include providing a manner of creating an opening in the conduit 112 of the ventilating system 270.

It will be appreciated that the opening 127 (FIG. 4) in the conduit 112 (FIGS. 3, 4 and 9) may be created in a variety of ways, including, for example, through the use of a trocar, a piercing member, scissors, a cutting instrument or the like. As will be appreciated, while any suitable way or manner of creating such an opening may be used, it is desirable that when creating such an opening 127 that the opening 127 is no larger than necessary to accommodate the portion or portions of the drain assembly 100 (FIGS. 3-5, 8 and 9) that are to be inserted therein. The use of openings larger than necessary to accommodate the portion or portions of the drain assembly 100 that are to be inserted into or through the opening 127 may cause or result in the leakage of the fluids from the conduit 112. As mentioned above; this type of leakage may be avoided or limited with the use of certain retention mechanisms 106 (see e.g., FIGS. 3 and 8) and/or seals 115 (FIG. 3).

In at least one embodiment a piercing member (not shown) may be used to create the opening 127. Further still, in a more specific embodiment, the conduit 114 (FIGS. 3, 4 and 8) may be sized so as to receive such a piercing member. The piercing member may be inserted into the conduit 114 in such a manner that the piercing member creates an opening in the conduit 114. Once the opening 127 (FIG. 4) is created the piercing member is removed and the reservoir 104 (FIGS. 3-5 and 8) may be attached to the lid 120 (FIGS. 3-5 and 8) which is secured to conduit 114.

It will be appreciated that most, if not all, embodiments of the present invention may be inserted into a conduit 112 (FIGS. 3, 4 and 9) at anytime; however, in order to minimize disruption to the circuit and/or to avoid leakage, it is generally desirable, when possible, to insert the drain at a time when the conduit 112 is not in use.

In those embodiments which utilize a retention mechanism, the method of the present invention may further include securing the assembly 100 the conduit 112. It will be appreciated that the manner in which the retention mechanism secures the assembly 100 to the circuit may vary depending upon the type of retention mechanism used as well as the type of conduit 112 used. While not inclusive, a number of different retention mechanisms and the way they can operate are discussed above.

As will be appreciated, the liquids which condense or “rain out” of the gases will tend to gather or accumulate at low points 272 (FIG. 9) along the ventilating system 270 (FIG. 9) and more specifically at low points along the conduits 112 (FIGS. 3, 4 and 9) leading to and from the patient 22 (FIG. 9). As such, the opening 127 (FIG. 4) in the conduit 112 is desirably created at a low point 272 along the conduits 112 as illustrated in FIG. 9. It will be appreciated that the drain assembly 100 need not be located at the lowest point along the conduits 112 (FIGS. 3, 4 and 9) of the system 270 (FIG. 9), but are desirably in close proximity thereto so as to maximize the amount of accumulated liquids that may be removed. It will also be appreciated that multiple drain assemblies 100 (FIGS. 3-5, 8 and 9) may provide for maximum liquid removal where numerous low points along the conduits 112 (FIGS. 3, 4 and 9) exist.

As suggested above, once the liquid has entered the drain assembly 100 (FIGS. 3-5, 8 and 9) it may become necessary to evacuate or otherwise empty the reservoir 104 (FIGS. 3-5 and 8). One way of emptying the reservoir 104 includes separation of the reservoir 104 from lid 120 (FIGS. 3-5 and 8) and the dumping of the liquids, or the replacement of the reservoir 104 with an another reservoir 104. However, in those embodiments of the present invention in which the lid 120 is more permanently secured to the reservoir 104 or in those instances in which it is desirable to for the attendant to avoid contact or the possibility of contact with the liquid therein, the liquid may be aspirated from the reservoir 104 in any number of suitable ways, some of which are discussed above in more detail. For instance, reservoir 104 (FIGS. 3-5 and 8) may include an aspiration valve or the like, such as evacuation pipe 132 (FIGS. 3-5 and 8). As discussed above, the point of access for the aspiration of liquids from the reservoir 104 is not crucial in that it may be in the lid 120 (FIGS. 3-5 and 8) of the reservoir 104, the side of the reservoir 104, and even in the bottom of the reservoir 104 in some embodiments. That being said, the method described above, may further include aspirating the accumulated liquid from the reservoir 104. It is contemplated that the step of aspirating may be continuous or periodical. The method could also include providing a mechanism for aspirating fluid 200 (see, for example, FIGS. 5 and 6) from the reservoir 104 (FIGS. 3-5 and 8). As noted above, it will be appreciated that the mechanism for aspirating the fluid from the reservoir 104 may include, but is not limited to, the suction sources and vacuum devices described above, as well as metered pumps or the like. In at least some embodiments the step of aspirating fluid from the reservoir 104 may include a probe such as that shown at 220 (FIGS. 5-7) which may be received by the reservoir 104 (FIGS. 3-5 and 8), a probe-receiving member 124 (FIGS. 3-5, 7 and 8) or a connection or fitting thereto.

As noted above, the present invention is also contemplated for use with a heated wire circuit. In those instances in which a drain assembly 100 (FIGS. 3-5, 8 and 9) of the present invention is used with a heated wire circuit the opening 127 (FIG. 4) should be created and the drain assembly secured to the conduit 112 (FIGS. 3, 4 and 9) so as not to interfere or disrupt the electrical/heating element 276 (FIG. 4) running therethrough.

It will be appreciated that unlike prior devices the present invention allows for the installation of a drain assembly 100 before or after fluids begin to flow through a conduit 112. Additionally, as noted above, the present invention need not be inserted between two conduits or a break (i.e. separation) in one conduit; rather the present invention may be inserted into a pre-existing conduit (as shown in FIG. 9 ). The ability to utilize the present invention provides the ability to install a drain assembly during use of the conduit with minimal or no interruption of the fluid flow through the conduit; whereas prior devices required the complete separation of conduits so as to allow the insertion of a member between the two pieces of conduit. Clearly, such separation would prevent fluid flow through the conduit during installation. Such an interruption will be undesirable, if not impermissible, under such circumstances. Furthermore, even when prior devices are able to be installed in a conduit prior to use, the need of prior devices to be inserted between conduits or pieces of a conduit creates multiple connection points, thereby increasing the opportunity for leaks in the system.

While the invention has been described in detail with respect to specific embodiments thereof, those skilled in the art, upon obtaining an understanding of the invention, may readily conceive of alterations to, variations of, and equivalents to the described embodiments and the processes for making them. It is intended that the present invention include such modifications and variations as come within the scope of the appended claims and their equivalents.

We claim:

1. A fluid collection device for collecting condensed vapor and moisture from a gas directing conduit having an opening created therein, the collection device comprising:
a lid member;
a reservoir capable of forming a seal with the lid member, the reservoir intended for receiving vapor and moisture; and
a duct having a proximal end and a distal end, the distal end being in communication with the lid member and the proximal end adapted for communication with the conduit.

2. The device of claim 1 wherein the duct is capable of sealingly engaging the opening in the conduit.

3. The device of claim 1, wherein the duct in communication with the lid member is integrally formed to the lid member.

4. The device of claim 1, wherein the duct is sized so as to be capable of receiving a piercing member.

5. The device of claim 1 further comprising a retention mechanism capable of retaining the duct in position relative to the conduit.

6. The device of claim 5 in which the retention mechanism encompasses a portion of the conduit so as to maintain the position of the device relative to the conduit.

7. The device of claim 5 in which the retention mechanism is at least partially inside the conduit when the device is properly positioned.

8. The device of claim 1, wherein the duct has one or more openings which enable fluid to flow from the conduit into the reservoir.

9. The device of claim 1 in which the reservoir has an access port to allow for the aspiration of fluid from the reservoir.

10. The device of claim 9 in which the access port is a normally closed opening in the reservoir capable of receiving a probe through which suction is selectively communicated so as to remove fluid from the reservoir.

11. The device of claim 9 in which the access port is a normally closed opening in the lid member capable of receiving a probe through which suction is selectively communicated so as to remove fluid from the reservoir.

12. The device of claim 10 wherein an evacuation tube is interposed between the normally closed opening of the reservoir and the interior of the reservoir.

13. The device of claim 10 wherein a suction tube is interposed between the normally closed opening of the lid member and the interior of the reservoir.

14. A method of draining fluid from a gas directing tube comprising:
providing a fluid collection device;
creating an opening in the gas directing tube; and
inserting at least a portion of the fluid collection device into the opening in the gas directing tube such that fluids within the gas directing tube may flow into the reservoir.

15. The method of claim 14 further comprising aspirating fluid from the reservoir.

16. The method of claim 15 in which the step of aspirating includes periodically aspirating accumulated fluid from the reservoir.

17. The method of claim 14 in which the fluid collection device comprises:
a lid member;
a reservoir capable of forming a seal with the lid member, the reservoir intended for receiving vapor and moisture; and
a conduit having a proximal end and a distal end, the distal end being in communication with the lid member and the proximal end adapted for communication with the gas directing tube.

18. A method of draining a heated wire circuit comprising:
providing a fluid collection device intended for use with a circuit having an opening created therein, the collection device comprising:
a lid member;
a reservoir capable of forming a seal with the lid member, the reservoir intended for receiving vapor and moisture; and
a conduit having a proximal end and a distal end, the distal end being in communication with the lid member and the proximal end adapted for communication with the circuit; and
a retention mechanism for maintaining the position of the assembly relative to the circuit;
creating an opening in the circuit such that at least a portion of the conduit may be inserted therein without interfering with the heating elements of the circuit; and
securing the assembly to the circuit such that fluids within the circuit may flow into the reservoir.

19. The method of claim 18, wherein the fluid collection device is inserted into the circuit at a low point along the circuit.
